(19) United States
(12) Patent Application Publication
(10) Pub. No.: US 2009/0133165 A1
(43) Pub. Date: May 21, 2009

(54) SEED DEVITALIZATION METHOD

(75) Inventors: Phillip Guy, Creve Coeur, MO (US); Charles J. Shank, Ballwin, MO (US)

Correspondence Address: SENNIGER POWERS LLP (MTC), 100 NORTH BROADWAY, 17TH FLOOR, ST LOUIS, MO, 63102, US

(73) Assignee: MONSANTO TECHNOLOGY LLC, St. Louis, MO (US)

(21) Appl. No.: 12/269,358

(22) Filed: Nov. 12, 2008

Related U.S. Application Data
(60) Provisional application No. 60/988,160, filed on Nov. 15, 2007.

(51) Int. Cl. A01H 5/10

(52) U.S. Cl. 800/298

(57) ABSTRACT

The present invention relates to a method for devitalizing seeds that provides a non-viable (i.e., non-germinating) seed exhibiting substantially the same protein and/or deoxyribonucleic acid (DNA) characteristics as a viable seed and also relates to devitalized seeds produced by the method.

### REFERENCE TO RELATED APPLICATIONS

This application claims the benefit of U.S. Provisional Application Ser. No. 60/988,160, filed Nov. 15, 2007, the entire contents of which are incorporated herein by reference.

### FIELD OF THE INVENTION

The present invention relates to a method for devitalizing seeds that provides a non-viable (i.e., non-germinating) seed exhibiting substantially the same protein and/or deoxyribonucleic acid (DNA) characteristics as a viable seed and also relates to devitalized seeds produced by the method.

### BACKGROUND OF THE INVENTION

Plant seeds are germinated as part of their growth cycle. Germination is defined by the American Organization of Seed Analysts (AOSA) as the emergence and development from the seed embryo of those essential structures which, for the kind of seed in question, are indicative of the ability to produce a normal plant under favorable conditions. Germination may be triggered by various environmental conditions (e.g., temperature, moisture, and oxygen). For example, corn seeds typically absorb about 30% of their weight in water before germination begins. This absorption of water by a seed to trigger germination is commonly referred to as imbibing the seed.

In some circumstances it may be desirable to eliminate the ability of a seed to germinate. Rendering a seed non-germinating is generally referred to as seed devitalization. Conventional devitalization methods generally involve subjecting the seed to elevated temperatures and/or moisture conditions, for example, in an autoclave.

Devitalized seeds may be desired in a variety of situations. For example, many jurisdictions have begun, or are expected to begin requiring whole samples of conventional and genetically modified seeds as part of their regulatory approval process. Submission of whole, viable seeds may be undesired since there is a risk of appropriation of valuable germplasm information and transgenic traits embodied in the seed. Thus, to protect germplasm and seed traits it would be beneficial to provide devitalized whole seed samples. Unfortunately, however, the conditions of conventional devitalization methods generally result in denaturating of seed protein and/or DNA and, therefore, are unacceptable for biochemical identification of DNA or protein from the seed, or to serve as reference material (i.e., standards) for regulatory purposes. In addition, elevated moisture contents of seeds devitalized by conventional methods may increase the risk of seed putrefaction.

Accordingly, there exists an unfulfilled need for a seed devitalization method that provides a non-viable seed exhibiting substantially the same protein and/or DNA characteristics as the viable seed prior to devitalization treatment. A further need exists for a seed devitalization method that does not increase the risk of seed putrefaction.

### SUMMARY OF THE INVENTION

Briefly, therefore, the present invention is directed to a method for preparing a devitalized seed, the method comprising contacting a viable seed with an aqueous medium, thereby initiating germination and producing an imbibed seed; and subjecting the imbibed seed to a temperature of less than about 0° C. to devitalize the imbibed seed.

The present invention is further directed to devitalized seeds prepared by the present method. In various embodiments, the present invention is directed to a devitalized seed produced from a viable seed, wherein the devitalized seed has a moisture content within about 3% of the moisture content of the viable seed. In various other embodiments, the present invention is directed to a devitalized seed produced from a viable seed, wherein the devitalized seed has protein and/or DNA characteristics substantially similar to those of the viable seed.

The present invention is further directed to devitalized seeds produced from viable seeds wherein the results of analysis by the following methods for the devitalized seeds and viable seeds are not statistically different at a 95% confidence level: quantitative polymerase chain reaction (qPCR) assays; qualitative polymerase chain reaction (PCR) assays; protein detection in accordance with an ELISA assay; and Single Nucleotide Polymorphism (SNP) assays.

Other objects and features will be in part apparent and in part pointed out hereinafter.

### BRIEF DESCRIPTION OF THE DRAWINGS

FIG. 1 is a standard curve of a plot of the log concentration generated as described in Example 1.

FIG. 2 is a plot of predicted lines generated as described in Example 1.

### DETAILED DESCRIPTION OF THE PREFERRED EMBODIMENTS

Described herein is a seed devitalization method that provides a devitalized seed that may be analyzed by conventional protein and DNA detection methods (e.g., to detect transgenes and expressed proteins) with results representative of the viable seed. That is, the devitalized seed exhibits substantially the same protein and/or DNA characteristics as the viable seed such that the devitalized whole seed is suitable for biochemical identification of protein or DNA from the seed as may be required for regulatory or other purposes. Generally, the method of the present invention comprises initiating seed germination by imbibing the seed through contact with an aqueous medium and then subjecting the imbibed seed to temperatures below about 0° C. to devitalize the seed. It is believed that imbibing the seed hydrates cells and organelles necessary to initiate seed germination and these hydrated cells and organelles are then at least partially destroyed by freezing, thereby rendering the seed non-viable. In this manner, the method of the present invention may be referred to as a “freeze-fracture” approach.

Advantageously, as described herein and detailed in the Examples, the present method does not substantially alter seed proteins or DNA. Accordingly, the method may be used to prepare non-viable seeds that are suitable for biochemical identification of protein or DNA from the seed, or to serve as reference material or a standard as required for various purposes (e.g., regulatory approval or genetic testing) while avoiding the risk of appropriation of valuable germplasm and/or transgenic traits. It is believed that the devitalized seeds prepared by the present method may generally qualify as plant material, rather than as plants, under the applicable regulations of various jurisdictions. Since regulations governing transmission of plants are more stringent than those governing transmission of plant material in most, if not all jurisdictions, this represents a further benefit of the present method.

The method of the present invention is generally suitable for devitalization of any type of seeds, notably principal field crop seeds such as corn, cotton, and soybean seeds.

Conventional devitalization methods typically increase the moisture content of the seed which increases the risk of seed putrefaction. Imbibing and subsequent cooling in accordance with the present method increases the moisture content of the seeds, but the risk of seed putrefaction is minimized, and preferably substantially eliminated by the present method since the increase in moisture content is accompanied by subjecting the seed to relatively cool (e.g., freezing) temperatures. Thus, the manner of devitalization of the present method reduces the risk of seed putrefaction. Furthermore, in accordance with a preferred embodiment, the moisture content of the devitalized seeds may be reduced to at or near typical seed storage moisture levels (e.g., initial viable seed moisture content) to provide a devitalized seed amenable for protein and/or DNA analysis that may be stored in a manner and for a duration similar to viable seeds. Thus, the present method may further reduce, and preferably substantially eliminate, issues of seed putrefaction sometimes associated with conventional devitalization methods.

Seed devitalization by the present method may also substantially reduce, and preferably substantially eliminate, the presence of seed-borne pathogens and phytosanitary concerns associated therewith during transport and/or storage of the seeds. Namely, reduced pathogen content reduces or eliminates the risk of propagation of diseases, viruses and other microorganisms between seeds during transport and/or storage that may occur with viable seeds. In addition, shipment of devitalized seeds as reference material may reduce or eliminate the possibility of disease propagation and/or plant-to-plant spread of disease that can be associated with transport and germination of viable seed.

### I. Imbibing

In accordance with the method of the present invention, the seed is contacted with an aqueous medium (e.g., distilled, or tap water) to initiate germination and produce an imbibed seed.

The conditions and manner of contact of the seed with the aqueous medium are not narrowly critical, but are generally selected to provide an imbibed seed that has initiated the process of germination. For example, a quantity of viable seeds may be placed in a liquid-permeable nylon bag and submerged in a bath of the aqueous medium containing sufficient liquid to imbibe the seeds to the desired moisture content.

While the imbibed seed of the present method has initiated germination, completion of germination to the extent of shoot (e.g., radicle) emergence from the seed (typically referred to as Phase 2 of water uptake) is avoided. The rate of seed germination generally increases with increasing temperature. Typically, the temperature of the aqueous medium is from about 5° C. to about 40° C. But to provide improved control with respect to avoiding shoot emergence, it is preferred that the temperature of the aqueous medium contacted with the seed is generally below about 20° C., preferably below about 15° C. and, more preferably, below about 10° C. However, imbibing may also be suitably conducted at higher temperatures (e.g., from about 25° C. to about 35° C.) with a concomitant increase in germination rate so long as measures are taken to avoid shoot emergence (e.g., by reducing contact time).

The time of contact between the seed and aqueous medium during imbibing will vary depending, in part, on the temperature of the bath. Typically, the seed and aqueous medium are contacted for at least about 1 hour, at least about 4 hours, at least about 12 hours, at least about 24 hours, or at least about 48 hours. Generally, the seed and aqueous medium are contacted with the aqueous medium for from about 1 to about 48 hours, from about 6 to about 36 hours, or from about 12 to about 24 hours. Suitable combinations of temperature and contact time may be selected such that an imbibed seed of the desired moisture content is obtained while avoiding shoot emergence. These combinations can be readily determined through trial and error by one skilled in the art. For example, in the case of corn seeds, a seed typically absorbs about 30% of its weight in moisture before it is sufficiently imbibed to initiate germination. Other types of seeds may absorb from about 20% to about 30% of their weight in moisture before being sufficiently imbibed to initiate germination. However, in accordance with the present method it is to be noted that the precise proportion of moisture absorbed by the imbibed seed relative to its initial weight is not narrowly critical, so long as the seed is sufficiently imbibed to initiate germination, but completion of Phase 2 of water uptake is avoided.

In various embodiments, the aqueous medium contacted with the seed consists essentially of water (e.g., distilled, or tap water). In still other embodiments, the aqueous medium may include an additive to reduce the population of bacteria, viruses, and/or fungi at the surface of the seed. For example, in various embodiments, the aqueous medium contacted with the seed contains chlorine ions. Typically, the aqueous medium contains less than about 20 wt %, less than about 10 wt %, or less than about 5 wt % chlorine ions.

In these and other embodiments, an aqueous medium contacted with the seed may contain an osmoticum to reduce the osmotic potential of the medium and promote control of water uptake by the seed. Suitable osmoticum may be selected from among those known in the art, including the group consisting of polyethylene glycol, mannitol, various polymers, and combinations thereof. The concentration of osmoticum in the aqueous medium is not narrowly critical and will generally be at a level that contributes to inhibiting onset of Phase 2 of water uptake by the seed.

Additionally or alternatively, the seed may be contacted with an aqueous medium containing an additive designed to reduce hardness of the seed and/or remove dormancy of the seed. Such an additive may be, for example, ethaphon, potassium nitrate, or a combination thereof. The concentration of an additive(s) for either or both of these purposes in the aqueous medium is not narrowly critical and can be readily determined by one skilled in the art. In accordance with these and various other embodiments, the seed may be subjected to a pretreatment of relatively short duration to break seed dormancy and/or reduce seed hardness. This pre-treatment generally comprises submerging the seed in an aqueous medium (with or without any of the above-noted additives) at temperatures of at least about 50° C., or at least about 60° C. for no more than about 10 minutes (e.g., no more than about 5 minutes, or no more than about 3 minutes).

The seed may first be contacted with a liquid medium containing one or more of the above-noted types of additives in a pre-treatment step for the primary purpose of providing one or more of the above-noted benefits (e.g., addressing phytosanitary concerns), rather than initiate germination of the seed. The total additive content in any pre-treatment or imbibing liquid medium is typically less than about 30 wt %, more typically less than about 25 wt % and, still more typically, less than about 20 wt % and can be readily determined through trial and error by one skilled in the art.

### II. Devitalization

The imbibing process described above initiates germination of the seed and hydrates seed cells and organelles. The seed is removed from the imbibing bath and subjected to temperatures below about 0° C. to terminate germination of the seed prior to shoot emergence. Without being bound to a particular theory, it is believed that the imbibed seeds are rendered non-viable once subjected to low temperatures for a period of time sufficient to freeze and at least partially destroy the hydrated cell walls and organelles. As noted, in this manner the method of the present invention may be referred to as “freeze-fracture” devitalization.

The conditions under which the imbibed seed is subjected to low temperature treatment are not narrowly critical, but are generally selected and/or controlled to damage the cellular material of the imbibed seed and produce a devitalized seed. For example, typically the imbibed seed is subjected to a temperature of less than about −10° C., less than about −20° C., less than about −30° C., less than about −40° C., less than about −50° C., less than about −60° C., less about −70° C., or less than about −80° C.

The time for which the imbibed seed is subjected to low temperature treatment is not narrowly critical and is dependent on the temperature employed. Typically, low temperature treatment of the imbibed seed proceeds for at least about 1 hour, at least about 2 hours, or at least about 4 hours. But, regardless of the temperature and duration of contact, these conditions are selected to freeze and at least partially destroy cell walls and organelles of the imbibed seed to an extent sufficient to produce a devitalized seed. Generally, the present method provides devitalization of at least about 90% of the seeds treated, typically at least about 95% and, still more typically, devitalization of at least about 99% of the seeds treated (e.g., 99.5% or greater devitalization). In accordance with a preferred embodiment, the freeze-fracture method provides 100% devitalization. For example, seed germination testing indicates devitalization (e.g., providing dead or non-germinated seeds) of 100 seed replicates in each of ten, 100 seed trials. The conditions of the low temperature treatment can be readily determined through trial and error by one skilled in the art.

In various embodiments the imbibed seed is subjected to low temperature treatment in a freezer suitable for this purpose. In still other embodiments, the imbibed seed may be subjected to freezing temperatures by virtue of contact with a super cooled fluid such as, for example, liquid nitrogen. Generally, treatment of the seed in this manner is carried out for less than about 15 minutes, less than about 10 minutes, or less than about 5 minutes.

### III. Moisture Reduction

As noted, since various conventional devitalization methods include increasing the moisture content of the seed, the devitalized seeds may be prone to putrefaction. Although the freeze-fracture method of the present invention increases the moisture content of the seed, as previously noted, this increase is coupled with relatively low, typically freezing temperatures that inhibit, and preferably substantially prevent seed putrefaction.

Furthermore, in accordance with a preferred embodiment, the moisture content of devitalized seeds produced by the freeze-fracture method may be reduced to at or near typical seed storage moisture levels (e.g., initial viable seed moisture contents). For example, commercial viable corn seeds typically contain from about 11% to about 12.5% by weight moisture; commercial viable cotton seeds typically contain from about 9% to about 11% by weight moisture; and commercial viable soybean seeds typically contain from about 9% to about 11% by weight moisture. This preferred embodiment provides a further benefit over conventional methods with regard to reduction in seed putrefaction risks and, since the devitalized seed has a moisture content at or near the initial viable seed moisture content, the seed is suitable for storage over longer periods of time. Generally, the moisture content of the devitalized seed is reduced to within about 3% of the initial viable seed moisture content, preferably reduced to within about 2% and, more preferably, reduced to within about 1% of the initial viable seed moisture content.

The devitalized seeds may be dried by various methods known in the art including, for example, passage of relatively dry air at various temperatures through the seed sample. The air temperature is not narrowly critical, but is generally maintained at a level that avoids denaturation of seed protein(s) of interest. For example, devitalized seeds may be contacted with air at a temperature of no more than about 40° C., no more than about 30° C., no more than about 25° C., or no more than about 20° C. to reduce seed moisture content to the desired level. In various other embodiments, the moisture content of devitalized seeds may be reduced by lyophilization (i.e., freeze-drying) in accordance with means known in the art. Regardless of the manner of drying, the moisture content of the devitalized seeds may be determined using methods and apparatus known in the art including, for example, dielectric methods practiced using a Model GAC II Grain Analysis Computer available from the Dickey-john Corporation.

However, it is to be understood that the freeze-fracture method provides a devitalized seed that is amenable to conventional protein and DNA detection methods without moisture reduction. For example, depending on the interval between seed devitalization and analysis, reduction in moisture content may be unnecessary.

Further in accordance with the present method, imbibed seed may be subjected to a freeze-drying operation to both devitalize the seed and provide a devitalized seed of suitable moisture content in a single operation by virtue of the lyophilizer providing a substantially moisture-free environment at a temperature less than about 0° C.

### IV. Devitalized Seed Characteristics

Advantageously, as noted above and detailed in the Examples, the freeze-fracture method does not substantially alter the protein and genomic DNA of the seed.

Biochemical identification analysis (e.g., by conventional protein and DNA detection methods) of devitalized seeds produced in accordance with the present invention has provided substantially similar results as those obtained from analysis of the viable seeds. Biochemical identification methods suitable for analyzing viable and devitalized seeds are generally known in the art and include, for example, (i) quantitative polymerase chain reaction (qPCR) assays, (ii) qualitative polymerase chain reaction (PCR) assays, (iii) protein strip tests; (iv) single seed enzyme linked immunosorbent assays (ELISA), and (v) Single Nucleotide Polymorphism (SNP) single seed assays to determine varietal purity. Results of analysis of corn, cotton, and/or soybean seeds by one or more of these analysis methods are set forth below in the Examples. In accordance with the present invention, it has been discovered that the results of one or more of these analysis methods for devitalized seeds produced by the present invention and for the corresponding viable seeds generally are not statistically different at a confidence level of 95%. More particularly, it is currently believed that the present method provides devitalized seeds that when subjected to various biochemical identification analysis methods provide results that are not statistically different from the results obtained from analysis of viable seeds at a 96% confidence level, at a 97% confidence level, at a 98% confidence level, or at a 99% confidence level.

It is to be noted that the similarity in protein and/or DNA characteristics of devitalized seeds of the present invention as compared to the corresponding viable seeds may be demonstrated by methods known in the art not listed herein or described in the following Examples. Moreover, the analytical similarities are currently not believed to depend on the particular conditions of the analysis methods employed. One skilled in the art can select an appropriate method and analysis conditions for comparison of the devitalized seeds and viable seeds depending on the particular situation (e.g., type of seed and/or transgene of interest).

The present invention is illustrated by the following examples which are merely for the purpose of illustration and not to be regarded as limiting the scope of the invention or the manner in which it may be practiced.

### EXAMPLES

### Example 1

This example describes a devitalization procedure conducted using corn seeds that is generally applicable to other types of seeds (e.g., cotton and soybean seeds). Once devitalized, the seeds were tested for germination using an American Organization of Seed Analysts (AOSA)/International Seed Testing Association (ISTA) sanctioned warm germination test. For comparison purposes, viable seeds were also tested.

The devitalized and viable seeds were also subjected to comparative analysis by a quantitative polymerase chain reaction (qPCR) assay to detect hmg (a single copy endogenous maize gene encoding a high mobility group protein).

Devitalization

A seed counter was used to identify the amount of seed to be tested, which was placed in a labeled nylon mesh bag. The seed-containing bag was submerged in a bucket of tap water so that the water level reached approximately 1 inch above the level of the seeds. The bucket was stored at approximately 10° C. for approximately 24 hours. The bucket was checked periodically to ensure that the seeds remained fully submerged in the water.

After approximately 24 hours of submersion, the bag was removed from the water and excess water was allowed to drain. The bag was then placed in a freezer at a temperature of approximately −20° C. and stored for approximately 16 hours. The frozen seed was then placed in a lyophilizer (Virtis Freeze Dryer, Model 360 DX66) and dried using conventional means known in the art. Seed moisture was checked periodically and the drying operation continued until the seed moisture content was approximately 12 wt %, as determined using a Grain Analysis Computer available from the Dickey-john Corporation.

Germination Testing

For the AOSA/ISTA warm germination test, the seeds were held on watered roll towels for between 4 and 7 days at 25° C. (+/−1° C.). The towels were evaluated to determine the number of normal seedlings, the number of abnormal seedlings and the number of devitalized seeds (e.g., abnormal seedlings or dead or non-germinated seeds). The results are shown in the following Table.

TABLE 1

| | Number of seeds | Number of seeds | % germination at |
|---|---|---|---|
| Sample | tested | germinated | 95% confidence |
| Viable | 1000 | 974 | ≧96.41 |
| Devitalized | 1000 | 0 | ≦0.30 |

DNA/qPCR Analysis

Six samples (three viable and three devitalized) of approximately 5 g each of conventional maize seed were extracted and purified for genomic DNA. Genomic DNA from conventional wheat was extracted and purified to be used as non-maize DNA backfill for generation of the standard curves.

A Hoefer Scientific fluorometer was used to quantify the DNA samples. Standard curves consisting of 8 points (100, 50, 10, 5, 1, 0.5, 0.1, and 0.05% maize DNA) were generated from the DNA obtained from each of the viable and devitalized maize samples (n=6).

A quantitative polymerase chain reaction (qPCR) assay designed to detect hmg (a single copy endogenous maize gene encoding a high mobility group protein) was performed. The hmg assay is a recognized as a maize-specific internal calibrator for quantitative Taqman® assays by the Community Reference Laboratory For GM Food & Feed of the European Commission Joint Research Centre.

To broadly assess the devitalization method, the endogenous maize gene hmg was used as the target in these qPCR experiments rather than a GM-specific assay.

Each standard curve (3 generated from the viable and 3 from the devitalized portion of the conventional maize lot) was analyzed in triplicate. To make statistical comparisons of the slopes for viable and devitalized seeds the following model was fit to the data:

Yᵢⱼ=μ+Tᵢ+βᵢXᵢⱼ+∈ᵢⱼ (1)
where, Yᵢⱼ=Ct of the j^{th} extraction for the i^{th} treatment (viable, devitalized); μ=The overall mean; Tᵢ=Effect of the i^{th} treatment; βᵢ=Slope of regression line for the i^{th} treatment; Xᵢⱼ=Log concentration of the j^{th} extraction for the i^{th} treatment; ∈ᵢⱼ=Residual effect; Ct is the cycle in the Taqman® assay in which the signal (fluorescence) generated by amplification of the target sequence surpasses the background fluorescence of the assay.

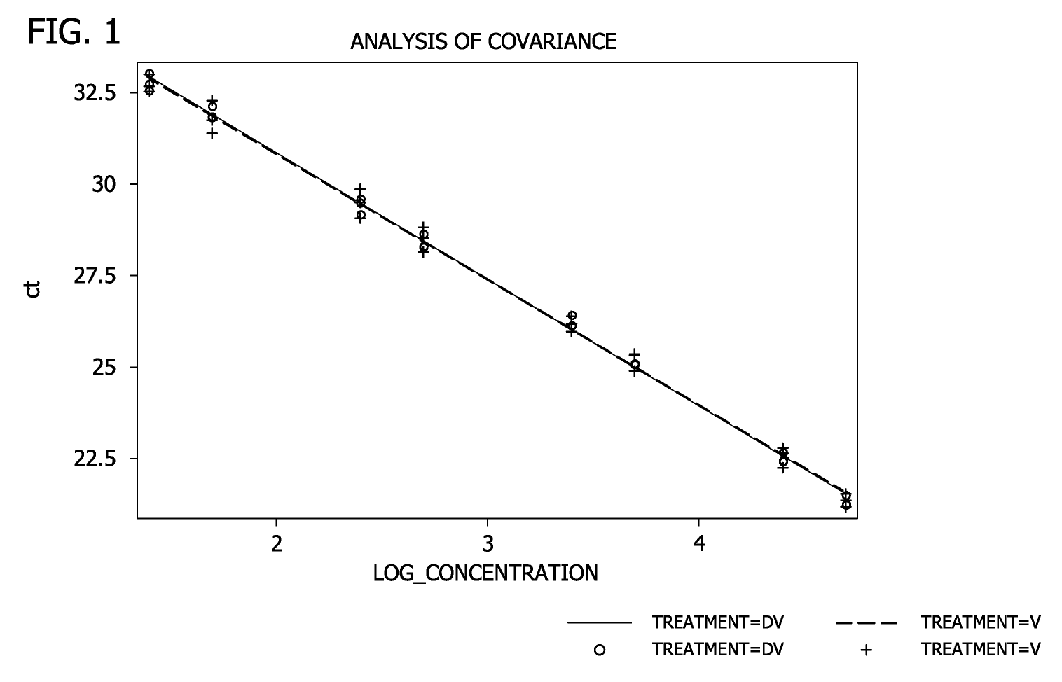

FIG. 1 includes the standard curve of a plot of the log concentration of the targeted sequence at each point on the standard curve (x axis) versus Ct (y axis) produced by model (1) for viable seeds. FIG. 1 also includes a plot of the log concentration of the targeted sequence at each point on the standard curve (x axis) versus Ct (y axis) for devitalized seeds. As shown in FIG. 1, the slopes of the curves for viable and devitalized seeds were not statistically different. More particularly, the slopes of the curves were not statistically different at a confidence level of at least 95%.

Table 2 displays the results of the comparison of the slopes of viable and devitalized standard curves (“Estimate” is the estimated difference between the slopes). The slopes were not significantly different at the 5% level.

TABLE 2

| Parameter | Estimate | Standard Error | t value | Pr > \|t\| |
|---|---|---|---|---|
| DV v. V slope | −0.02760878 | 0.06375484 | −0.43 | 0.6671 |

DV = Devitalized, V = Viable

Since the slopes were not significantly different a simplified equal slopes model of the following form was fit to the data:

Yᵢⱼ=β+Tᵢ+βXᵢⱼ+∈ᵢⱼ (2)
where, Yᵢⱼ=Ct of the j^{th} extraction for the i^{th} treatment (viable, devitalized); μ=The overall mean; Tᵢ=Effect of the i^{th} treatment; β=Slope of regression lines; Xᵢⱼ=Log concentration of the j^{th} extraction for the i^{th} treatment; ∈ᵢⱼ=Residual effect.

Table 3 displays the results of the comparison of the intercepts of viable and devitalized standard curves (“Estimate” is the estimated difference between the intercepts). The intercepts were not significantly different at the 5% level.

TABLE 3

| Parameter | Estimate | Standard Error | t value | Pr > \|t\| |
|---|---|---|---|---|
| DV v. V | −0.00208333 | 0.07127087 | −0.03 | 0.9768 |
| intercept | | | | |

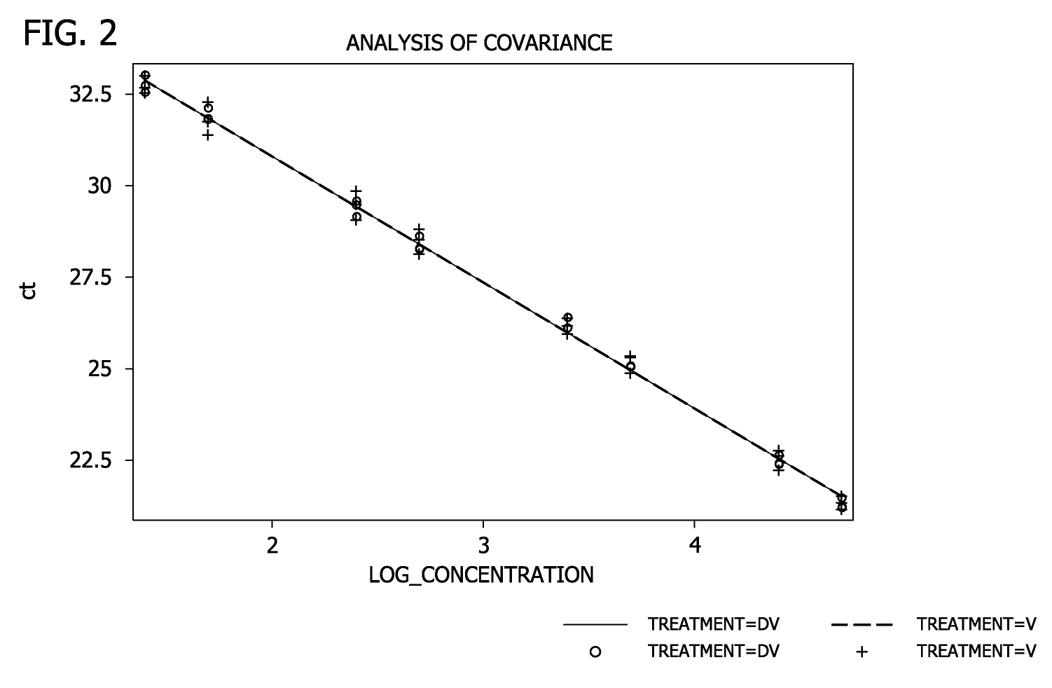

FIG. 2 is a plot of the predicted lines produced by model (2). As shown in FIG. 2, the standard curves for DNA extracted from devitalized and viable seeds were not statistically different when analyzed by qPCR, indicating the devitalization process does not negatively impact DNA behavior.

### Example 2

This Example details germination, protein, and DNA analysis of corn seeds devitalized in accordance with the method described in Example 1 to determine the impact of devitalization on corn seeds containing three known traits.

Seed germination/viability was determined using the AOSA/ISTA sanctioned warm test procedure referred to in Example 1. Germination results are set forth in Table 4.

TABLE 4

| | Pre-Devitalized | Devitalized |
|---|---|---|
| Sample | Germination (%) | Germination (%) |
| 1 | 98.75 | 0 |
| 2 | 98.50 | 0 |
| 3 | 99.50 | 0 |
| 4 | 92.00 | 0 |
| 5 | 94.25 | 0 |
| 6 | 99.00 | 0 |
| 7 | 99.00 | 0 |
| 8 | 98.50 | 0 |
| 9 | 99.25 | 0 |

Prior to devitalization, germination ranged between 92 and 99.5% for the 400 viable seeds tested. After devitalization, all samples showed zero normal seedlings in 1000 seeds per sample giving a 95% confidence that viability is below 0.3%, estimated to be 0% germination.

Seeds tested included three hybrids of viable and devitalized seeds, each hybrid containing one of the three traits. Each of the 9 (viable and devitalized) hybrids across the three traits was tested for three replications. Analysis was conducted to detect the presence of genes corresponding to three traits: (1) glyphosate resistance (RR) (cp4 epsps), (2) corn rootworm resistance (CRW) (cry3Bb1), and (3) YIELDGARD corn rootworm resistance (YG) (cry1Ab).

Viable and devitalized seeds containing each trait were analyzed by each of three methods: (1) analysis for presence of the expressed transgene/trait by an End-point Taqman qualitative polymerase chain reaction (PCR) assay; (2) detection of the protein expressed by the gene of interest using a single seed ELISA assay; and (3) analysis using a Single Nucleotide Polymorphism (SNP) single seed assay to determine varietal purity of viable and devitalized seeds. The results for each method are shown in Table 5.

TABLE 5

| | | Devitalized Seed | | | | | Viable Seed | | | | |
|---|---|---|---|---|---|---|---|---|---|---|---|
| | | PCR | Zero | ELISA | Zero | SNP | PCR | Zero | ELISA | Zero | SNP |
| Trait | Sample | + ve | DNA | + ve | Protein | Purity (%) | + ve | DNA | + ve | Protein | Purity (%) |
| RR | 1 | 78.3 | 1.3 | 87.3 | 0.0 | 95.9 | 79.0 | 0.7 | 85.7 | 0.0 | 97.4 |
| RR | 2 | 77.0 | 2.3 | 87.3 | 0.3 | 97.8 | 78.7 | 1.3 | 87.3 | 0.0 | 97.4 |
| RR | 3 | 80.0 | 0.0 | 87.0 | 0.0 | 99.6 | 78.7 | 0.7 | 87.7 | 0.0 | 98.9 |
| CRW | 4 | 78.3 | 1.0 | 87.0 | 0.0 | 98.2 | 79.3 | 0.0 | 86.3 | 0.0 | 98.9 |
| CRW | 5 | 79.3 | 0.0 | 87.7 | 0.3 | 98.5 | 79.3 | 0.0 | 87.7 | 0.3 | 97.8 |
| CRW | 6 | 77.7 | 1.3 | 87.7 | 0.0 | 96.7 | 78.0 | 0.0 | 87.0 | 0.7 | 96.3 |
| YG | 7 | 79.7 | 0.0 | 88.0 | 0.0 | 99.6 | 79.3 | 0.3 | 87.3 | 0.0 | 98.9 |
| YG | 8 | 80.0 | 0.0 | 87.0 | 0.0 | 100.0 | 80.0 | 0.0 | 87.3 | 0.3 | 100.0 |
| YG | 9 | 78.7 | 1.0 | 86.3 | 0.0 | 98.2 | 77.7 | 1.7 | 87.3 | 0.0 | 96.3 |

Statistical analysis (Fisher's Exact Test) showed no significant differences for the results for viable and devitalized seeds analyzed by each of these methods.

Three, 80 seed replicates were analyzed by the PCR assay. The table provides the average number of seeds over the three replicates in which the gene of interest was detected (PCR+ve), and the average number of seeds in which the gene of interest was not detected (Zero DNA). The results for detection of the gene of interest for devitalized seeds were generally within about 2.5%, or less, of the detection results for the viable seeds and were not statistically different at a confidence level of at least 95%.

Three, 88 seed replicates were analyzed by the ELISA single seed assay to detect expression of protein by the gene of interest (ELISA+ve) or absence of expressed protein (Zero Protein). Protein detection results averaged over the three replicates for the viable and devitalized seeds were generally within about 1.5% and were not statistically different at a confidence level of at least 95%.

Three, 90 seed replicates were analyzed by the SNP single seed assay to detect varietal purities of both viable and devitalized seeds with reference to a known standard. The varietal purities for viable and devitalized seeds were not statistically different at a confidence level of at least 95%. The purity of the viable seeds ranged from 96.3 to 100%; the purity for the devitalized seeds ranged from 95.9% to 100%.

### Example 3

This example details germination testing and qualitative polymerase chain reaction (PCR) analysis of viable cotton seeds and cotton seeds devitalized in accordance with the present method.

Cotton seed was devitalized generally in accordance with the method described in Example 1, except the seed was imbibed for approximately 48 hours. Viable and devitalized seeds were tested for germination by the AOSA/ISTA method detailed in Example 1. Eight, 50 seed replicates of viable seeds were tested. Twenty, 50 seed replicates of devitalized seeds were tested. The results are shown in Table 6.

TABLE 6

| Pre-Devitalized | Devitalized |
|---|---|
| Germination (%) | Germination (%) |
| >90 | 0 |

Prior to devitalization, germination was greater than 90%. After devitalization, zero normal seedlings were observed in the 1000 seeds, indicating a confidence level of 95% that viability is below 0.3%, estimated to be 0%.

Viable and devitalized seeds were analyzed by a qualitative polymerase chain reaction (PCR) assay; three, 80 seed replicates of viable and devitalized seeds were analyzed. The PCR assay detects the presence of three endogenous cotton genes (cp4 epsps, cry1Ac, and cry2Ab).

Protein analysis was conducted utilizing a single seed ELISA assay; three, 80 seed replicates were analyzed.

Average results of the PCR and ELISA assays for the three replicates are shown in Table 7.

TABLE 7

| | Devitalized Seed | | Viable Seed | |
|---|---|---|---|---|
| Trait | PCR % + ve | ELISA % + ve | PCR % + ve | ELISA % + ve |
| 1 | 99.5 | 100 | 100 | 97.4 |
| 2 | N/A | 99.6 | N/A | 98.1 |

Detection levels for the PCR and ELISA assays varied only slightly between viable and devitalized seed. More particularly, the results for viable and devitalized seeds were not statistically different at a confidence level of at least 95%.

### Example 4

This example details germination testing and quantitative polymerase chain reaction (qPCR) analysis of viable soybean seeds and soybean seeds devitalized in accordance with the present method.

Soybean seeds were devitalized generally in accordance with the method described in Example 1, except the seeds were imbibed for approximately 6 hours. Viable and devitalized seeds were tested for germination using the AOSA/ISTA method described in Example 1.

Results of the germination testing were as follows:

TABLE 8

| | Number of seeds | Number of seeds | % germination at |
|---|---|---|---|
| Sample | tested | germinated | 95% confidence |
| Viable | 800 | 769 | 94.81 |
| Devitalized | 800 | 0 | 0.37 |

Viable and devitalized soybean seeds were also analyzed by quantitative PCR (qPCR). Six samples (three viable and three devitalized) of approximately 5 g each of conventional soybean seed were extracted and purified for genomic DNA. Genomic DNA from conventional wheat was extracted and purified to be used as non-soybean DNA backfill for generation of the standard curves. A Hoefer Scientific fluorometer was used to quantify the DNA samples. Standard curves consisting of 8 points (100, 50, 10, 5, 1, 0.5, 0.1, and 0.05% soybean DNA) were generated from the DNA obtained from each of the viable and devitalized soybean samples (n=6).

A qPCR assay designed to detect lectin (lec), a soybean endogenous gene was performed. The lec assay has been validated as a soybean-specific internal calibrator for quantitative Taqman® assays by the Community Reference Laboratory of the Joint Research Centre, part of the European Commission.

To make statistical comparisons of the slopes for viable and devitalized seeds the following model was fit to the data:

Yᵢⱼ=μ+Tᵢ+βᵢXᵢⱼ+∈ᵢⱼ (1)
where, Yᵢⱼ=Ct of the j^{th} extraction for the i^{th} treatment (viable, devitalized); μ=The overall mean; Tᵢ=Effect of the i^{th} treatment; ∈ᵢ=Slope of regression line for the i^{th} treatment; Xᵢⱼ=Log₁₀ concentration of the j^{th} extraction for the i^{th} treatment; ∈ᵢⱼ=Residual effect; Ct is the cycle in the Taqman® assay in which the signal (fluorescence) generated by amplification of the target sequence surpasses the background fluorescence of the assay.

TABLE 9
Comparison of the slopes for viable and devitalized seed:

| Difference | Standard Error | t-value | P-value |
|---|---|---|---|
| −0.046 | 0.106 | −0.43 | 0.6679 |

Because the slopes were not significantly different, a simplified equal slopes model of the following form was fit to the data to compare intercepts of the regression lines:

Yᵢⱼ=β+Tᵢ+βXᵢⱼ+∈ᵢⱼ (2)
where, Yᵢⱼ=Ct of the j^{th} extraction for the i^{th} treatment (viable, devitalized); μ=The overall mean; Tᵢ=Effect of the i^{th} treatment; β=Slope of regression lines; Xᵢⱼ=Log₁₀ concentration of the j^{th} extraction for the i^{th} treatment; ∈ᵢⱼ=Residual effect.

Table 10 displays the results of the comparison of the intercepts for viable and devitalized. The intercepts were significantly different at the 5% level.

TABLE 10

| Difference | Standard Error | t-value | P-value |
|---|---|---|---|
| −0.245 | 0.119 | −2.07 | 0.0445 |

The following table compares the viable and devitalized models' ability to quantify, back transformed inverse predictions were done, for a Ct of 27 (approximately corresponds to a 5% sample), using both models. The difference is expressed in terms of percent of the viable inverse prediction.

TABLE 11

| | Vitalized | Devitalized | Percent |
|---|---|---|---|
| Ct | Quantification | Quantification | Difference |
| 27 | 3277.21 | 2764.06 | −15.66 |

Because the intercepts were significantly different, a model of the following form was fit to the data to further investigate the source of the significant difference:

Yᵢ=μ+Tᵢ+βXᵢ+∈ᵢ (3)
where, Yᵢ=Ct of the i^{th} extraction and treatment (viable, devitalized) combination; μ=The overall mean; Tᵢ=Effect of the i^{th} extraction and treatment (viable, devitalized) combination; β=Slope of regression lines; Xᵢ=Log₁₀ concentration of the i^{th} extraction and treatment (viable, devitalized) combination; ∈ᵢ=Residual effect.

The following is an ABC plot of the results for all pairwise intercept comparisons at the 5% level.

TABLE 12

| Line | Estimate | Group |
|---|---|---|
| DS*2 | 38.13 | A |
| VS*3 | 38.18 | A |
| DS*3 | 38.21 | A |
| VS*1 | 38.87 | B |
| DS*1 | 38.92 | B |
| VS*2 | 38.95 | B |

(DS = Devitalized Soybean, VS = Viable Soybean)

These results indicate that the slopes of the models for viable and devitalized seed were not significantly different at the 5% level. These results also indicate that the intercepts of the models for viable and devitalized seed were significantly different at the 5% level. Thus, these results based on the soybean gene, lec, indicate that the devitalization method detailed herein did not negatively impact DNA analysis for devitalized soybean seeds as compared to analysis for viable seeds.

### Example 5

This example details quantitative polymerase chain reaction (qPCR) analysis of viable cotton seeds and cotton seeds devitalized in accordance with the present method. Cotton seeds were devitalized generally in accordance with the method described in Example 3 and seed germination assessed as described in Example 3. Prior to imbibing for 48 hours as described in Example 3, the seed was subjected to pretreatment by submerging in water at approximately 62° C. for approximately 3 minutes.

6 samples (3 viable and 3 devitalized) of approximately 5 g each of conventional cotton seed were extracted and purified for genomic DNA. Genomic DNA from conventional wheat was extracted and purified to be used as a non-cotton DNA backfill for generation of the standard curves. A Hoefer Scientific fluorometer was used to quantify the DNA samples. Standard curves consisting of 8 points (100, 50, 10, 5, 1, 0.5, 0.1, and 0.05% cotton DNA) were generated from the DNA obtained from each of the viable and devitalized seeds (n=6). For both viable and devitalized seed, the three independent extractions were run in triplicate at each point on the standard curve. The means of the triplicate runs were taken for each point and used in the analysis.

A qPCR assay was used to detect acp1, an endogenous cotton gene that encodes an acyl carrier protein. A cotton-specific reference was used which amplifies a 76-bp fragment of acp1. Amplification utilizes a pair of acp1 gene-specific primers and an acp1 gene-specific probe labeled with 6-FAM and TAMRA. This assay has been validated as a cotton-specific internal calibrator for quantitative Taqman® assays by the Community Reference Laboratory of the Joint Research Centre, part of the European Commission.

For the analysis using model (3) a new variable “Line” was created by combining the variables treatment and extraction.

The data were supplied as an EXCEL file, and were read into SAS (V9.1.3) for statistical analysis under Windows XP. (SAS Software Release 9.1 (TS1M3). Copyright 2002-2003 by SAS Institute Inc., Cary N.C.) Statistical Model and Analysis:

To make statistical comparisons of the slopes for viable and devitalized seed the following model was fit to the data:

Yᵢⱼ=μ+Tᵢ+βᵢXᵢⱼ+∈ᵢⱼ (1)
where, Yᵢⱼ=Ct of the j^{th} extraction for the i^{th} treatment (viable, devitalized); μ=The overall mean; Tᵢ=Effect of the i^{th} treatment; βᵢ=Slope of regression line for the i^{th} treatment; Xᵢⱼ=Log₁₀ concentration of the j^{th} extraction for the i^{th} treatment; ∈ᵢⱼ=Residual effect; Ct is the cycle of the Taqman® assay in which the signal (fluorescence) generated by the amplification of the target sequence surpasses the background fluorescence of the assay.

Table 13 provides a comparison of the slopes for viable and devitalized seeds. The slopes were not significantly different at the 5% level.

TABLE 13

| Difference | Standard Error | t-value | P-value |
|---|---|---|---|
| −0.013 | 0.067 | −0.19 | 0.8507 |

Because the slopes were not significantly different, a simplified equal slopes model of the following form was fit to the data to compare intercepts of the regression lines:

Yᵢⱼ=β+Tᵢ+βXᵢⱼ+∈ᵢⱼ (2)
where, Yᵢⱼ=Ct of the j^{th} extraction for the i^{th} treatment (viable, devitalized); μ=The overall mean; Tᵢ=Effect of the i^{th} treatment; β=Slope of regression lines; Xᵢⱼ=Log₁₀ concentration of the j^{th} extraction for the i^{th} treatment; ∈ᵢⱼ=Residual effect.

Table 14 displays the results of the comparison of the intercepts for viable and devitalized seeds. The intercepts were significantly different at the 5% level.

TABLE 14

| Difference | Standard Error | t-value | P-value |
|---|---|---|---|
| −0.229 | 0.075 | −3.05 | 0.0038 |

To compare the viable and devitalized models' ability to quantify, back transformed inverse predictions were done, for a Ct of 27, using both viable and devitalized models. The difference is expressed in terms of percent of the viable inverse prediction. The results are displayed in the following table. The difference in the inverse predictions is expressed in terms of percent of the viable inverse prediction.

TABLE 15

| | Viable | Devitalized | Percent |
|---|---|---|---|
| Ct | Quantification | Quantification | Difference |
| 27 | 2841.57 | 2419.34 | −14.86 |

Because the intercepts were significantly different, a model of the following form was fit to the data to further investigate the source of the significant difference:

Yᵢ=μ+Tᵢ+βXᵢ+∈ᵢ (3)
where, Yᵢ=Ct of the i^{th} extraction and treatment (viable, devitalized) combination; μ=The overall mean; Tᵢ=Effect of the i^{th} extraction and treatment (viable, devitalized) combination; β=Slope of regression lines; Xᵢ=Log₁₀ concentration of the i^{th} extraction and treatment (viable, devitalized) combination; ∈ᵢ=Residual effect.

Table 16 is an ABC plot of the results for all pairwise intercept comparisons at the 5% level. Only one of the three extractions of devitalized seed was significantly different. (DC=Devitalized Cotton; VS=Viable Cotton)

TABLE 16

| Line | Estimate | Group |
|---|---|---|
| DC*1 | 37.75 | A |
| DC*2 | 38.23 | B |
| VC*3 | 38.25 | B |
| DC*3 | 38.30 | B |
| VC*2 | 38.35 | B |
| VC*1 | 38.36 | B |

### CONCLUSION

The slopes of the models for viable and devitalized seed were not significantly different at the 5% level. The intercepts of the models for viable and devitalized seed did show a significant difference at the 5% level which was due to one extraction (extraction 1) of devitalized seed being significantly different from the other extractions. This was deemed to be within acceptable error and due to slight variations in the extraction efficiency between samples.

Overall, these results indicate that DNA extracted from devitalized seed is not practically different from DNA extracted from viable seeds.

The present invention is not limited to the above embodiments and can be variously modified. The above description of the preferred embodiments, including the Examples, is intended only to acquaint others skilled in the art with the invention, its principles, and its practical application so that others skilled in the art may adapt and apply the invention in its numerous forms, as may be best suited to the requirements of a particular use.

With reference to the use of the word(s) comprise or comprises or comprising in this entire specification (including the claims below), unless the context requires otherwise, those words are used on the basis and clear understanding that they are to be interpreted inclusively, rather than exclusively, and applicants intend each of those words to be so interpreted in construing this entire specification.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

1: A method for preparing a devitalized seed, the method comprising:
contacting a viable seed with an aqueous medium, thereby initiating germination and producing an imbibed seed; and
subjecting the imbibed seed to a temperature of less than about 0° C. to devitalize the imbibed seed.

2: The method as set forth in claim 1 comprising reducing the moisture content of the devitalized seed.

3: The method as set forth in claim 2 wherein the moisture content of the devitalized seed is within about 3% of the moisture content of the viable seed.

4: The method as set forth in claim 2 wherein the moisture content of the devitalized seed is reduced by subjecting the devitalized seed to a temperature of less than about 0° C. in a moisture-free environment.

5: The method as set forth in claim 1 wherein the temperature of the aqueous medium contacted with the viable seed is from about 5° C. to about 40° C.

6: The method as set forth in claim 1 wherein the temperature of the aqueous medium contacted with the viable seed is below about 20° C.

7: The method as set forth in claim 1 wherein the seed is contacted with the aqueous medium for at least about 4 hours.

8: The method as set forth in claim 1 wherein the aqueous medium comprises chlorine ions.

9: The method as set forth in claim 8 wherein the concentration of chlorine ions in the aqueous medium is less than about 30 wt %.

10: The method as set forth in claim 1 wherein the imbibed seed is subjected to a temperature of less than about 0° C. for at least about 1 hour.

11: The method as set forth in claim 1 wherein the imbibed seed is subjected to a temperature of less than about −10° C.

12: A devitalized seed produced from a viable seed, the devitalized seed having a moisture content within about 3% of the moisture content of the viable seed.

13: A devitalized seed produced from a viable seed, the devitalized seed having protein and DNA characteristics substantially similar to those of the viable seed.

14: A devitalized seed produced from a viable seed, wherein gene detection results of a quantitative polymerase chain reaction (qPCR) assay for the devitalized seed and the viable seed are not statistically different at a 95% confidence level.

15: The devitalized seed of claim 14, wherein gene detection results of a qPCR assay for the devitalized seed and the viable seed are not statistically different at a 96% confidence level.

16: A devitalized seed produced from a viable seed, wherein gene detection results of a qualitative polymerase chain reaction (PCR) assay for the devitalized seed and the viable seed are not statistically different at a 95% confidence level.

17: The devitalized seed of claim 16, wherein gene detection results of a PCR assay for the devitalized seed and the viable seed are not statistically different at a 96% confidence level.

18: A devitalized seed produced from a viable seed, wherein protein detection results of an ELISA assay for the devitalized seed and the viable seed are not statistically different at a 95% confidence level.

19: The devitalized seed of claim 18, wherein protein detection results of an ELISA assay for the devitalized seed and the viable seed are not statistically different at a 96% confidence level.

20: A devitalized seed produced from a viable seed, wherein varietal purity results of a Single Nucleotide Polymorphism (SNP) assay for the devitalized seed and the viable seed are not statistically different at a 95% confidence level.

21: The devitalized seed of claim 20, wherein varietal purity results of an SNP assay for the devitalized seed and the viable seed are not statistically different at a 96% confidence level.
